(19) United States
(12) Patent Application Publication
(10) Pub. No.: US 2022/0087587 A1
(43) Pub. Date: Mar. 24, 2022

(54) INFORMATION PROCESSING APPARATUS AND NON-TRANSITORY COMPUTER READABLE MEDIUM

(71) Applicant: FUJIFILM BUSINESS INNOVATION CORP., Tokyo (JP)

(72) Inventors: Masafumi KUDO, Kanagawa (JP); Junji HANATANI, Kanagawa (JP)

(73) Assignee: FUJIFILM BUSINESS INNOVATION CORP., Tokyo (JP)

(21) Appl. No.: 17/164,006

(22) Filed: Feb. 1, 2021

(30) Foreign Application Priority Data
Sep. 23, 2020 (JP) 2020-158715

(51) Int. Cl. A61B 5/22; G06T 7/70; G06T 7/20; A61B 5/395; A61B 5/11; A61B 5/00

(52) U.S. Cl. CPC A61B 5/224; G06T 7/70; G06T 7/20; A61B 5/395; A61B 2562/028; A61B 5/1116; A61B 5/743; A61B 5/6801; G06T 2207/30196; A61B 5/1107

(57) ABSTRACT

An information processing apparatus includes: a preparation device configured to prepare information on a posture of a body; a myoelectric potential meter configured to measure a myoelectric potential from a surface of the body; and a processor configured to acquire the information on the posture prepared by the preparation device, acquire information on the myoelectric potential measured by the myoelectric potential meter, specify a movement of the body based on the acquired information on the posture, estimate a muscle activity state required for a muscle to implement the specified movement, and output information indicating a difference between (i) a muscle activity state determined based on the myoelectric potential and (ii) the estimated muscle activity state.

### CROSS-REFERENCE TO RELATED APPLICATIONS

This application is based on and claims priority under 35 USC 119 from Japanese Patent Application No. 2020-158715 filed Sep. 23, 2020.

### BACKGROUND

### (i) Technical Field

The present disclosure relates to an information processing apparatus and a non-transitory computer readable medium.

### (ii) Related Art

With aging of skilled technicians, a transfer of their skills is an urgent issue. In addition, in order to improve an efficiency of sports practice, it is contemplated to extract characteristics of body movements of athletes. Thus, various methods are tried to analyze body movements of the technicians, the athletes, and the like.

For example, WO 2005/122900 discloses a method and a device that calculate a physically and physiologically appropriate muscle tension based on a musculoskeletal model. JP-A-2003-244027 discloses a muscle activity amount measurement device that estimates a muscle activity amount in a body by such adjustment that a surface myoelectric potential measured by surface electrodes arranged in a ring shape around an approximately cylindrical part of the body matches a surface myoelectric potential simulation value. JP-A-2017-159103 discloses a muscle activity audible method that outputs an acoustic signal representing information obtained from a relationship among plural values derived from plural myoelectric potentials.

### SUMMARY

It is known that people unconsciously put strength into a body during movements such as various skills and sports. However, it is difficult to measure such unconscious strength directly.

Aspects of non-limiting embodiments of the present disclosure relate to estimating unconscious strength in a movement of a person under measurement based on the movement and a muscle activity state.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including: a preparation device configured to prepare information on a posture of a body; a myoelectric potential meter configured to measure a myoelectric potential from a surface of the body; and a processor configured to acquire the information on the posture prepared by the preparation device, acquire information on the myoelectric potential measured by the myoelectric potential meter, specify a movement of the body based on the acquired information on the posture, estimate a muscle activity state required for a muscle to implement the specified movement, and output information indicating a difference between (i) a muscle activity state determined based on the myoelectric potential and (ii) the estimated muscle activity state.

### BRIEF DESCRIPTION OF THE DRAWINGS

Exemplary embodiment(s) of the present disclosure will be described in detail based on the following figures, wherein:

FIG. 1 is a diagram showing an example of a configuration of an information processing apparatus 1;

FIG. 2 is a diagram showing devices connected via an interface 13;

FIG. 3 is a diagram showing an example of a musculoskeletal model DB 121;

FIG. 4 is a diagram showing an example of a movement DB 122;

FIG. 5 is a schematic diagram showing a bone moved by muscles;

FIG. 6 is a diagram showing an example of a myoelectric potential DB 123;

FIG. 7 is a diagram showing an example of information on a relationship between a myoelectric potential and a muscle tension;

FIG. 8 is a diagram showing an example of a functional configuration of the information processing apparatus 1;

FIG. 9 is a flowchart of an example of an operation of the information processing apparatus 1;

FIG. 10 is a diagram showing an example of a change with time of an activity amount measured and estimated by the information processing apparatus 1;

FIG. 11 is a diagram showing an example of a change with time of an activity amount difference when a skilled worker is measured; and

FIG. 12 is a diagram showing an example of a change with time of an activity amount difference when a beginner is measured.

### DETAILED DESCRIPTION

### Exemplary Embodiment

### Configuration of Information Processing Apparatus

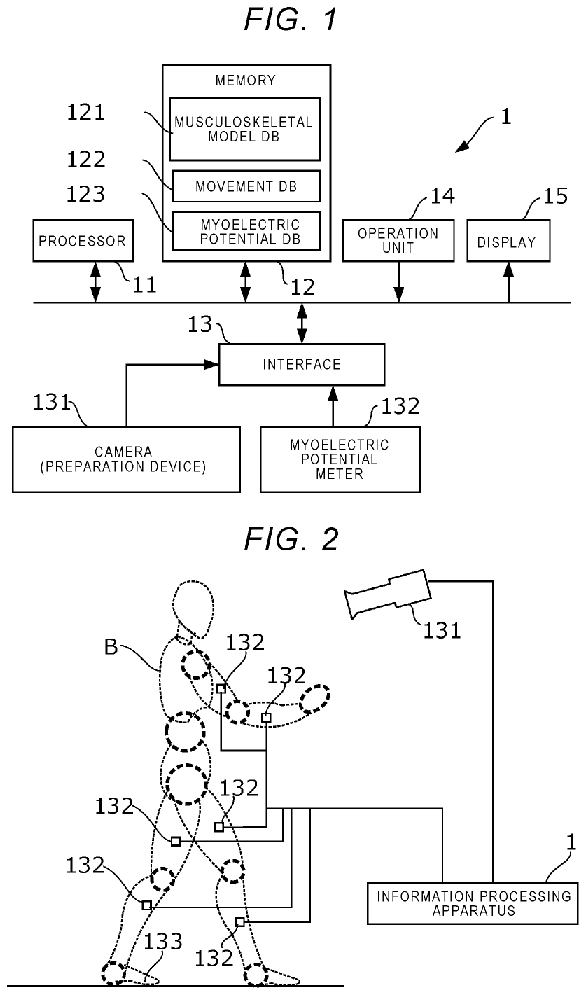

FIG. 1 is a diagram showing an example of a configuration of an information processing apparatus 1. The information processing apparatus 1 shown in FIG. 1 includes a processor 11, a memory 12, an interface 13, an operation unit 14, and a display 15. These units are communicably connected to each other via, for example, a bus.

The processor 11 controls each unit of the information processing apparatus 1 by reading and executing a program stored in the memory 12. The processor 11 is, for example, a central processing unit (CPU).

The operation unit 14 includes an operation element (such as operation buttons, a keyboard, a mouse, and a touch panel) for giving various instructions. The operation unit 14 receives an operation, and transmits a signal to the processor 11 according to an operation content thereof.

The display 15 displays a designated image under control of the processor 11. The display 15 shown in FIG. 1 includes a liquid crystal display which is a display screen for displaying the above image. A transparent touch panel of the operation unit 14 may be superposed on the liquid crystal display.

The interface 13 connects various devices to the processor 11 and causes the processor 11 to control those devices. The interface 13 shown in FIG. 1 connects a camera 131 and a myoelectric potential meter 132 to the processor 11.

FIG. 2 is a diagram showing devices connected via the interface 13. The camera 131 is a digital still camera including an optical system such as a lens (not shown) and an image capturing device such as a charge coupled device (CCD) or a complementary metal oxide semiconductor (CMOS).

The camera 131 shown in FIG. 2 captures an image of a body B of a person under measurement and generates image data indicating the captured image. Then, the camera 131 supplies the generated image data to the processor 11 via the interface 13. The processor 11 extracts a contour of the body B from the image indicated by the image data acquired from the camera 131, and generates information on a posture of the body B. The processor 11 extracts the contour of the body B from the image, using, for example, an edge detection algorithm such as the Canny method. The processor 11 specifies the posture of the body B by applying a machine learning algorithm such as a convolutional neural network to the extracted contour of the body B.

Here, the “information on a posture” may be a position and an orientation of a head, shoulders, arms, torso, and feet of the person under measurement. The camera 131 is a video camera that periodically captures the body B. In this case, the processor 11 specifies a change in the posture of the body B from plural images captured periodically. That is, the camera 131 is an example of a preparation device that prepares information on the posture of the body B by capturing an appearance of the body B and generating image data indicating the appearance of the body B.

The camera 131 is not limited to the digital still camera as long as the camera 131 is a device that prepares the information on the posture of the body B. For example, the preparation device may be a contact type micro electro mechanical systems (MEMS) sensor or the like instead of the camera 131. In this case, the contact type MEMS sensors are attached to plural parts that do not affect the work on the body B of the person under measurement, and acceleration or the like of those parts is measured. Then, this contact type MEMS sensor may supply information such as the measured acceleration to the processor 11 as the “information on a posture”.

The myoelectric potential meters 132 shown in FIG. 2 are, for example, myoelectric potential sensors, and are attached to a surface of the body B of the person under measurement. The myoelectric potential meters 132 measure myoelectric potentials of muscles under a skin at attached positions and supply signals indicating the myoelectric potentials to the processor 11. That is, the myoelectric potential meters 132 are examples of a myoelectric potential meter that measures the myoelectric potentials from the surface of the body.

The memory 12 shown in FIG. 1 is a storage that stores an operating system, various programs, data, and the like to be read into the processor 11. The memory 12 includes a random access memory (RAM) and a read only memory (ROM). The memory 12 may include a solid state drive, a hard disk drive, or the like. The memory 12 also stores a musculoskeletal model DB 121, a movement DB 122, and a myoelectric potential DB 123.

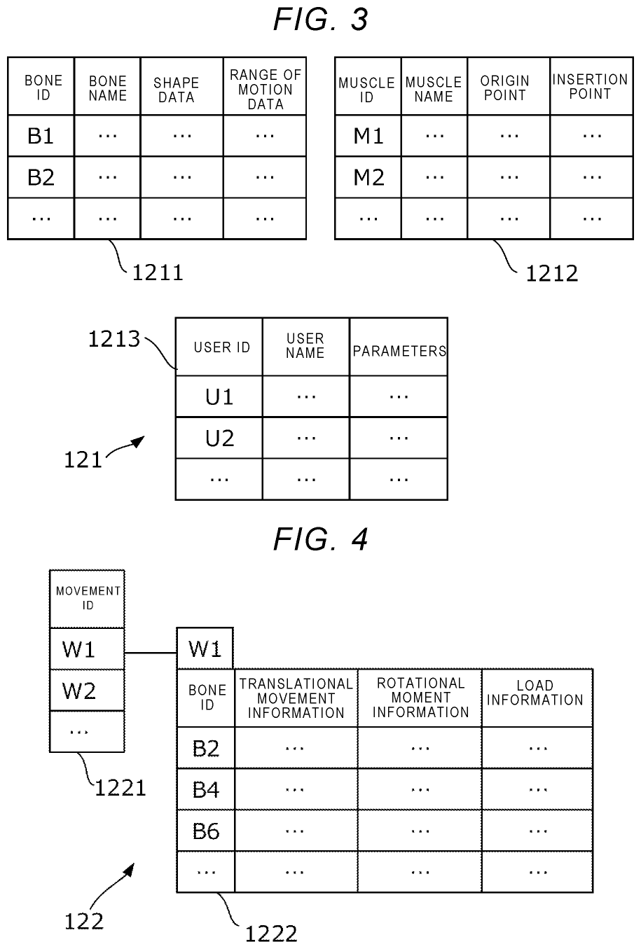

FIG. 3 is a diagram showing an example of the musculoskeletal model DB 121. The musculoskeletal model DB 121 is a database that stores physical models of bones and muscles. The musculoskeletal model DB 121 shown in FIG. 3 includes a bone model table 1211, a muscle model table 1212, and a user table 1213.

The bone model table 1211 is a table that describes information on the bones of the human body. The bone model table 1211 shown in FIG. 3 includes items of a bone ID, a bone name, shape data, and a range of motion data. The bone ID is identification information that identifies a bone of the human body. The bone name is a name of a bone identified by a corresponding bone ID. The shape data is data obtained by quantifying a shape of a bone identified by a corresponding bone ID in a three-dimensional space. The range of motion data is data that quantifies a range (referred to as a “range of motion”) where a bone identified by a corresponding bone ID moves. The information processing apparatus 1 specifies, for example, shapes or ranges of motion of bones of a standard human body with reference to the bone model table 1211.

The muscle model table 1212 is a table that describes information on the muscles of the human body. The muscle model table 1212 shown in FIG. 3 includes items of a muscle ID, a muscle name, an origin point, and an insertion point. The muscle ID is identification information that identifies a muscle of the human body. The muscle name is a name of a muscle identified by a corresponding muscle ID. The origin point is a position of a bone to which the muscle identified by the corresponding muscle ID is attached, and is a point where the muscle is attached to the bone that does not move in response to a movement of the muscle. The insertion point is a position of the bone to which the muscle identified by the corresponding muscle ID is attached, and is a point where the muscle is attached to the bone that moves due in response to a movement of the muscle. There may be plural origin points and plural insertion points for one muscle. The information processing apparatus 1 specifies, for example, a position of a bone to which a muscle of a standard human body is attached and a bone moved by the muscle with reference to the muscle model table 1212.

The user table 1213 is a table that describes information on bones and muscles unique to each user who is the person under measurement. The user table 1213 shown in FIG. 3 includes items of a user ID, a user name, and parameters. The user ID is identification information that identifies each person under measurement. The user name is a name of a user identified by a corresponding user ID. The parameters refer to various numerical values indicating bones and muscles unique to the user identified by the corresponding user ID.

The parameters may include, for example, a factor by which the shape data of the bone is multiplied to calculate a size of the bone of an individual user. Further, the parameters may include, for example, a maximum value of a tension (referred to as a “muscle tension”) output by the muscle of the individual user.

The parameters may be numerical values, nominal scales, or the like that indirectly indicate the information on the bones and the muscles of the user. For example, the parameters may include information such as a gender, an age, and genetic characteristics of the user. The information processing apparatus 1 specifies, for example, (i) characteristics such as laterality and strain of the bones and the muscles of the user and (ii) an ability such as an instantaneous force and endurance, with reference to the user table 1213, the bone model table 1211, and the muscle model table 1212 described above.

FIG. 4 is a diagram showing an example of the movement DB 122. The movement DB 122 is a database that stores, for each movement of the body, information on a bone that moves in response to the movement. The movement DB 122 shown in FIG. 4 stores a movement ID list 1221 and a bone movement table 1222.

The movement ID list 1221 lists movement IDs which are identification information for identifying movements performed by the person under measurement. Each bone movement table 1222 is stored in association with a corresponding one of the movement IDs included in the movement ID list 1221.

The bone movement table 1222 is a table that stores one or more bones moved by a movement identified by the corresponding movement ID and information on a movement of each bone when the bone moves. In the bone movement table 1222, the bone IDs are identification information common to the bone IDs in the above musculoskeletal model DB 121, and identify the bones of the human body.

Translational movement information is information on a translational movement applied to the bone identified by the corresponding bone ID. The translational movement is indicated in a combination of an x-axis direction, a y-axis direction, and a z-axis direction. Rotational moment information is information on a rotational moment applied to the bone identified by the corresponding bone ID. The rotational moment is indicated in a combination of a yaw, a pitch, and a roll.

Load information is information on a load exerted on the bone identified by the corresponding bone ID. The load may include gravity derived from a mass of the bone itself. Further, for example, when the person under measurement holds an object such as a dumbbell, a racket, or a bat by hands in the above movement, the load may include gravity derived from a mass of such an object.

For example, when the movement ID indicating the movement performed by the person under measurement is designated, the information processing apparatus 1 extracts the bone movement table 1222 corresponding to the movement ID with reference to the movement DB 122. The information processing apparatus 1 specifies shapes and masses of the bones identified by the bone IDs described in the extracted bone movement table 1222, and masses of the muscles that adhere to and move the bones. Then, the information processing apparatus 1 refers to the above musculoskeletal model DB 121 and calculates the muscle tensions generated in the muscles when these bones perform the movement indicated by the translational movement information and the rotational moment information while the load indicated by the load information exerts on the bones.

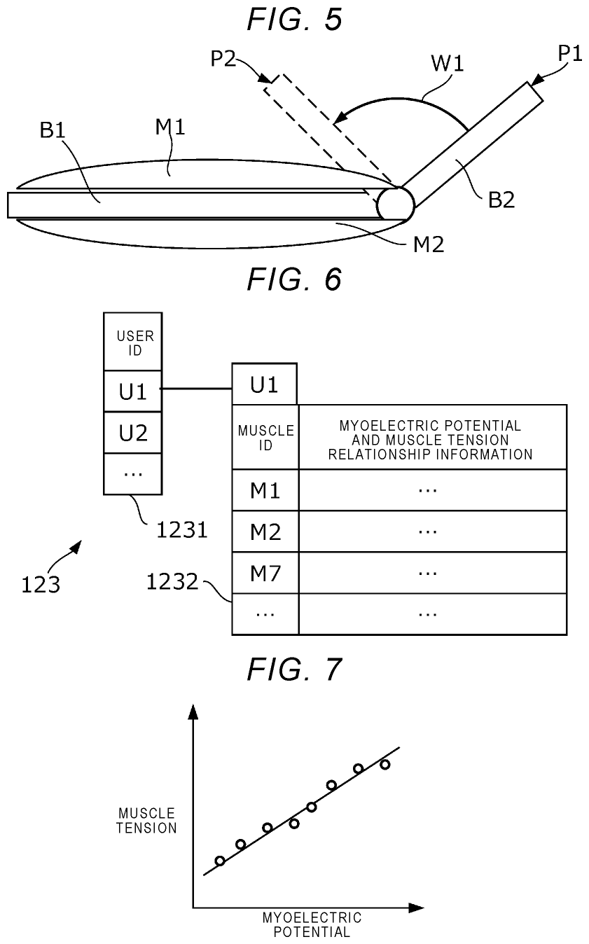

There may be many combinations of muscle tensions that muscles needs to produce in order to achieve a movement. FIG. 5 is a schematic diagram showing a bone moved by muscles. A bone B1 and a bone B2 shown in FIG. 5 are jointed at end portions thereof. Each of a muscle M1 and a muscle M2 is a muscle having an origin point in the bone B1 and a insertion point in the bone B2. Each of the muscle M1 and the muscle M2 moves the bone B2 with respect to the bone B1. The muscle M1 and the muscle M2 are so-called paired antagonist muscles. For example, the bone B1 is a humerus, the bone B2 is an ulna, the muscle M1 is a biceps brachii, and the muscle M2 is a triceps brachii.

When the muscle M1 contracts and the muscle M2 relaxes, the bone B2 moves from a position P1 to a position P2 (this movement will be referred to as a “movement W1”). This movement W1 is implemented, for example, by an action of a force of 7 Newtons in an arrow direction shown in FIG. 5. At this time, if the muscle M2 relaxes while applying a force of 3 Newtons in a direction opposite to a contraction direction of the muscle M1, the muscle M1 needs to apply a force in the arrow direction described above so as to (i) offset the force applied by the muscle M2 and (ii) apply the force of 7 Newtons to the bone B2. In this case, the force that the muscle M1 needs to generate is 10 Newtons, which is 7 Newtons plus 3 Newtons.

The information processing apparatus 1 calculates a minimum muscle tension that needs to be applied to those bones based on the movements of the bones that constitute the movement. For example, in the example shown in FIG. 5, the information processing apparatus 1 calculates the minimum muscle tension required for the muscle M1 to implement the movement W1 on an assumption that the muscle M2 relaxes without preventing the contraction of the muscle M1 at all. In this case, the force that muscle M1 needs to generate is only 7 Newtons.

FIG. 6 is a diagram showing an example of the myoelectric potential DB 123. The myoelectric potential DB 123 is a database that stores, for each user, a relationship between the myoelectric potential generated in each muscle and the muscle tension corresponding to the myoelectric potential. The myoelectric potential DB 123 shown in FIG. 5 stores a user ID list 1231 and a relationship table 1232.

The user ID list 1231 lists user IDs. The user IDs listed in the user ID list 1231 are identification information common to the user IDs in the musculoskeletal model DB 121 described above, and identify the user who is the person under measurement. Each relationship table 1232 is stored in association with a respective one of the user IDs included in the user ID list 1231.

The relationship table 1232 is a table that stores, for each muscle of the user identified by the corresponding user ID, information showing a relationship between the myoelectric potential generated in the muscle and the muscle tension exerted by the muscle when the myoelectric potential is generated. The muscle IDs in the relationship table 1232 are identification information common to the muscle IDs in the musculoskeletal model DB 121 described above, and identify the muscles of the body of the person under measurement.

Information on a relationship between a myoelectric potential and a muscle tension in the relationship table 1232 is stored in association with the muscle ID, and includes data showing the relationship between the myoelectric potential and the muscle tension which are generated in the muscle identified by the muscle ID. FIG. 7 is a diagram showing an example of the information on the relationship between the myoelectric potential and the muscle tension. The information on the relationship between the myoelectric potential and the muscle tension in the relationship table 1232 stores, for example, calibration curve data shown in FIG. 7.

That is, the information on the relationship between the myoelectric potential and the muscle tension may be a set including (i) a measured value of the myoelectric potential generated in the muscle and (ii) a measured value of the muscle tension exerted by the muscle when the myoelectric potential is generated. Alternatively, the information on the relationship between the myoelectric potential and the muscle tension may be a calibration curve specified from the set of these measured values. The information processing apparatus 1 specifies, for each user and each muscle, the relationship between the myoelectric potential and the muscle tension in the muscle with reference to the relationship table 1232 of the myoelectric potential DB 123, and for example, calculates the myoelectric potential corresponding to the muscle tension.

### Functional Configuration of Information Processing Apparatus

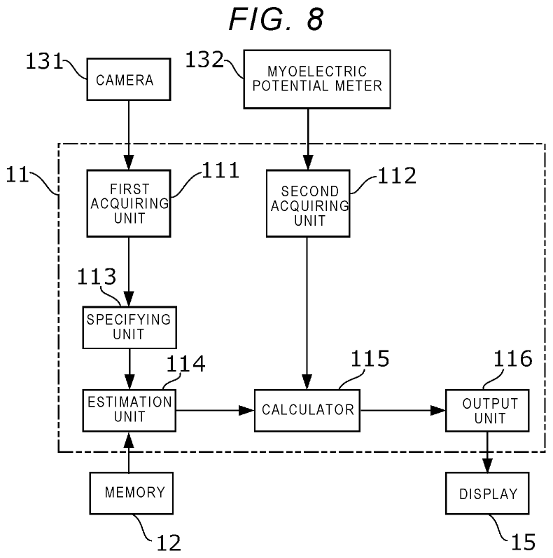

FIG. 8 is a diagram showing an example of a functional configuration of the information processing apparatus 1. The processor 11 of the information processing apparatus 1 serves as a first acquiring unit 111, a second acquiring unit 112, a specifying unit 113, an estimation unit 114, a calculator 115, and an output unit 116 by executing the program stored in the memory 12.

The first acquiring unit 111 acquires the information on the posture of the body B prepared by the camera 131 which is the example of the preparation device. The information acquired by the first acquiring unit 111 is, for example, image data indicating images periodically captured by the camera 131.

The second acquiring unit 112 acquires information on the myoelectric potentials measured by the myoelectric potential meter 132.

The specifying unit 113 specifies the movement of the body based on the information on the posture acquired by the first acquiring unit 111.

The estimation unit 114 estimates a minimum activity state required for a muscle to implement the movement of the body specified by the specifying unit 113. The estimation is performed by referring to the musculoskeletal model DB 121, the movement DB 122, and the myoelectric potential DB 123 stored in the memory 12.

The calculator 115 calculates a numerical value indicating a difference between (i) the muscle activity state determined based on the information on the myoelectric potentials acquired by the second acquiring unit 112 and (ii) the minimum activity state of the muscle estimated by the estimation unit 114. The muscle activity state determined based on the information on the myoelectric potentials may be the muscle tension or the myoelectric potential itself. The “numerical value indicating the difference” calculated by the calculator 115 is an example of information indicating the difference.

The output unit 116 outputs the numerical value indicating the difference calculated by the calculator 115 by displaying the numerical value on the display 15.

### Operation of Information Processing Apparatus

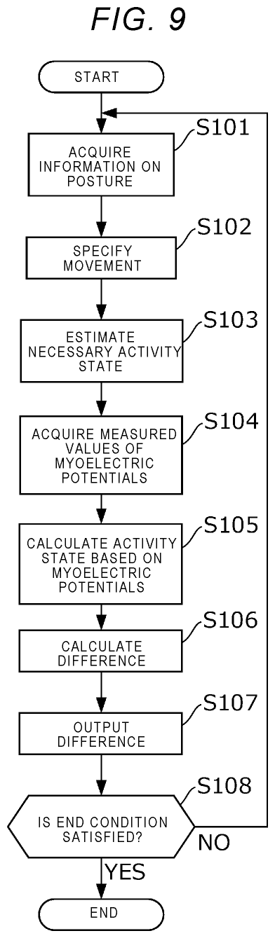

FIG. 9 is a flowchart of an example of a movement of the information processing apparatus 1. The processor 11 of the information processing apparatus 1 acquires the information on the posture from the camera 131 via the interface 13 (step S101). Then, the processor 11 specifies the movement performed by the person under measurement based on the acquired information on the posture (step S102). For example, the processor 11 derives the movement of each part (that is, each of the bones, the muscles, and the like) of the body of the person under measurement based on the change with time of the information on the posture, and thereby specifies the movement of the person under measurement.

After specifying the movement performed by the person under measurement, the processor 11 estimates the minimum activity state required for the muscle to implement the movement (step S103).

The processor 11 acquires measured values of the myoelectric potentials from the myoelectric potential meter 132 via the interface 13 (step S104). Then, the processor 11 calculates the muscle activity state of the person under measurement based on the acquired measured values of the myoelectric potentials (step S105).

The processor 11 calculates the difference between the muscle activity state calculated in step S105 and the minimum activity state of the muscle estimated in step S103 (step S106) and outputs the difference (step S107).

FIG. 10 is a diagram showing an example of a change with time of an activity amount measured and estimated by the information processing apparatus 1. In a graph shown in FIG. 10, a horizontal axis represents time, and a vertical axis represents the activity amount. The information processing apparatus 1 acquires information indicating the posture of the person under measurement and information on the myoelectric potentials when the person under measurement performs a series of the movement W1, a movement W2, and a movement W3 in this order.

For example, the movement W1 is a movement in which a person under measurement in a standing posture holds an object having a predetermined mass with a hand, moves the forearm without moving the humerus, and lifts the object to a height of the elbow. For example, the movement W2 is a movement in which the person under measurement is stationary while holding the object at the height of the elbow. For example, the movement W3 is a movement in which the person under measurement extends the forearm and lowers the object grasped by the hand downward. The hand is an end portion of the forearm.

A curve A0 is a curve showing the change with time of the estimated value of the minimum activity amount required for the muscle of the person under measurement to implement the above series of movements. The processor 11 of the information processing apparatus 1 reads the information on the bones and the muscles unique to the person under measurement from the musculoskeletal model DB 121, and constructs a physical model thereof. Then, the processor 11 analyzes the image data acquired from the camera 131 to specify the series of movements of the person under measurement, and analyzes the movements to calculate a direction and a magnitude of a minimum force required to implement this movement.

The processor 11 assigns the calculated direction and the magnitude of the force to each muscle of the person under measurement, and estimates the minimum activity amount required for each muscle. This activity amount may be, for example, the myoelectric potential representing one muscle or the muscle tension generated by the muscle.

A curve A1 is a curve showing the change with time of the activity amount of the muscle of the person under measurement, which is calculated from the measured values such as the myoelectric potentials measured when the series of movements are actually performed. The person under measurement at this time is a skilled worker in this movement.

The processor 11 of the information processing apparatus 1 acquires the information on the myoelectric potentials of the person under measurement from the myoelectric potential meter 132 via the interface 13. Then, the processor 11 calculates the activity amount of the muscle of the person under measurement based on the acquired information on the myoelectric potentials.

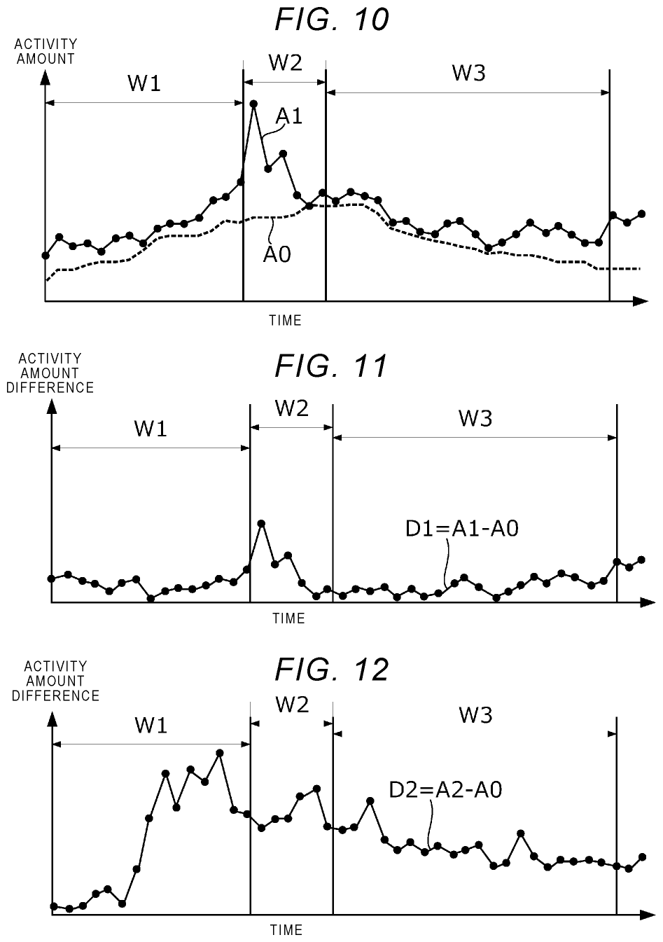

FIG. 11 is a diagram showing an example of a change with time of an activity amount difference when the skilled worker is measured. In a graph shown in FIG. 11, the horizontal axis represents the time, and the vertical axis represents an activity amount difference. The information processing apparatus 1 calculates a difference (which will be referred to as the “activity amount difference”) between (i) an actual activity amount of the muscle with the above movement by the skilled worker and (ii) the activity amount that is estimated by the physical model and that is minimum required for the muscle to implement the movement, and displays the change with time of the activity amount difference on the display 15 by a graph.

At this time, as shown in FIG. 11, the display 15 displays a difference D1 between (i) the curve A1 representing the activity amount determined based on the measured myoelectric potential of the muscle of the person under measurement who is the skilled worker and (ii) the curve A0 representing the estimated value of the minimum activity amount of the muscle required to implement the series of movements performed by the person under measurement (D1=A1−A0).

Here, the activity amount may be the myoelectric potential itself. In this case, the processor 11 of the information processing apparatus 1 is an example of a processor configured to estimate a myoelectric potential required for the muscle to implement the movement, and display, in a graph form, a change with time of a difference between (i) the myoelectric potential acquired from the myoelectric potential meter 132 and (ii) the estimated myoelectric potential.

FIG. 12 is a diagram showing an example of a change of time of an activity amount difference when a beginner is measured. In a graph shown in FIG. 12, the horizontal axis represents the time, and the vertical axis represents the activity amount difference. The person under measurement, who is the beginner, that is, is not skilled in the series of movements described above, has a longer period in which he unconsciously puts strength into his body than the skilled worker, and the magnitude of the unconscious strength is relatively large. Therefore, the information processing apparatus 1 displays a difference D2 shown in FIG. 12. The difference D2 is a difference between (i) a curve A2 (not shown) representing an activity amount determined based on a myoelectric potential obtained by measuring a muscle of the beginner and the curve A0 described above (D2=A2−A0).

In general, “unconscious strength” reduces a speed of a movement, limits a range of motion, and therefore the “unconscious strength” is an inhibition factor of the movement thereof. However, the “unconscious strength” is also an element necessary to control the body and perform the movement thereof accurately. Therefore, skilled movements are often characterized by a timing, a magnitude, or the like of the “unconscious strength”.

From the displayed difference D1 shown in FIG. 11 and the displayed difference D2 shown in FIG. 12, it is possible to know (i) a timing at which the skilled worker puts strength into his body when performing a predetermined movement and (ii) how much strength the skilled worker puts into his body when performing the predetermined movement. In addition, a magnitude of the unconscious strength and a timing of the unconscious strength when the beginner performs a common movement are apparent from the displayed difference D1 shown in FIG. 11 and the displayed difference D2 shown in FIG. 12. Therefore, by comparing a result of the beginner with a result of the skilled worker, the beginner can learn the magnitude of the unconscious strength and the timing of the unconscious strength that he is to be aware of in order to master this movement.

The information processing apparatus 1 may display the activity amount difference in the movement of the skilled worker and the activity amount difference in the movement of the beginner, and allow the user who is the beginner to compare. The information processing apparatus 1 may also calculate and display a difference between the activity amount in the movement of the skilled worker and the activity amount in the movement of the beginner.

The processor 11 determines whether a predetermined end condition is satisfied, for example, the operation unit 14 receives an end instruction from the user (step S108). When the processor 11 determines that the end condition is not satisfied (step S108, NO), the processor 11 returns the processing to step S101. On the other hand, when the processor 11 determines that the end condition is satisfied (step S108, YES), the processor 11 ends the processing.

By the operation, the information processing apparatus 1 estimates and outputs the unconscious strength in the movement of the person under measurement based on the movement and the muscle activity state. Therefore, the user of the information processing apparatus 1 knows an unconscious strength component in the movement of the person under measurement by distinguishing the unconscious strength from a minimum muscle tension required for the movement.

### MODIFICATIONS

The above is the description of the exemplary embodiment, and this exemplary embodiment may be modified as follows. In addition, the following modifications may be combined with each other.

<1>

In the above exemplary embodiment, the information processing apparatus 1 includes the processor 11 configured with the CPU. Alternatively, a controller that controls the information processing apparatus 1 may have another configuration. For example, the information processing apparatus 1 may include various processors or the like in addition to the CPU.

In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

<2>

In the embodiments above, the term “processor” is broad enough to encompass one processor or plural processors in collaboration which are located physically apart from each other but may work cooperatively.

The order of operations of the processor is not limited to one described in the embodiments above, and may be changed.

<3>

In the above exemplary embodiment, the processor 11 displays, in the graph form, the change with time of the difference between the acquired myoelectric potential and the estimated myoelectric potential. Alternatively, the processor 11 may output a timing at which this difference satisfies a predetermined condition. For example, when obtaining the change with time of the activity amount difference shown in FIG. 11, the processor 11 may notify the user of the timing at which the activity amount difference satisfies the predetermined condition. Here, the predetermined condition may be, for example, a condition that the calculated activity amount difference exceeds a threshold value, or a condition that the activity amount difference passes a local maximum value or a local minimum value.

<4>

In the above exemplary embodiment, the information processing apparatus 1 includes the display 15 that displays information indicating the difference between (i) the muscle activity state determined based on the myoelectric potentials and (ii) the estimated muscle activity state, to the user. Alternatively, the information processing apparatus 1 may include another output device. For example, the information processing apparatus 1 may include an output device that is attached to a part of the body of the user and that stimulates the part of the body.

In this case, when the processor 11 of the information processing apparatus 1 calculates the above difference, the processor 11 may control this output device and stimulate a part corresponding to a muscle related to this difference with a strength corresponding to the magnitude of this difference. In this case, the processor 11 is an example of a processor configured to, when generating the information indicating the difference between (i) the muscle activity state determined based on the myoelectric potentials on the user and (ii) the estimated muscle activity state, control an output device to stimulate a part corresponding to the muscle with a strength corresponding to a magnitude of the difference indicated by the generated information.

With this configuration, the body of the user is stimulated using the data obtained by measuring the skilled worker, so that the user can know (i) the part into which the skilled worker puts strength in the movement, (ii) the magnitude of the strength, and (iii) the timing at which the skilled worker puts the strength.

<5>

In the above exemplary embodiment, the information processing apparatus 1 includes the interface 13, and the camera 131 and the myoelectric potential meter 132 which are connected via the interface 13. The information processing apparatus 1 may not include these devices. In this case, these devices may be communicably connected to the information processing apparatus 1 via the interface 13 as external devices of the information processing apparatus 1.

That is, the processor 11 of the information processing apparatus 1 is an example of a processor provided in an information processing apparatus, the processor being configured to acquire information on a posture of a body, acquires information on a myoelectric potential on a surface of the body, specify a movement of the body based on the acquired information on the posture, estimate a muscle activity state required for a muscle to implement the specified movement, and output information indicating a difference between (i) a muscle activity state determined based on the myoelectric potential and (ii) the estimated muscle activity state.

<6>

In the above exemplary embodiment, the program executed by the processor 11 of the information processing apparatus 1 is an example of a program that causes a computer including a processor to execute: acquiring the information on a posture of a body; acquiring information on a myoelectric potential measured from a surface of the body; specifying a movement of the body based on the acquired information on the posture; estimating a muscle activity state required for a muscle to implement the specified movement; and outputting information indicating a difference between (i) a muscle activity state determined based on the myoelectric potential and (ii) the estimated muscle activity state.

This program may be provided in a state of being stored in a computer readable recording medium, such as a magnetic recording medium (for example, a magnetic tape and a magnetic disk), an optical recording medium (for example, an optical disc), an magneto-optical recording medium, and a semiconductor memory. Further, the program may be downloaded via a communication line such as the Internet.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a preparation device configured to prepare information on a posture of a body;
a myoelectric potential meter configured to measure a myoelectric potential from a surface of the body; and
a processor configured to
acquire the information on the posture prepared by the preparation device,
acquire information on the myoelectric potential measured by the myoelectric potential meter,
specify a movement of the body based on the acquired information on the posture,
estimate a muscle activity state required for a muscle to implement the specified movement, and
output information indicating a difference between (i) a muscle activity state determined based on the myoelectric potential and (ii) the estimated muscle activity state.

2. The information processing apparatus according to claim 1, wherein
the processor is configured to
estimate a myoelectric potential required for the muscle to implement the movement, and
display, in a graph form, a change with time of a difference between (i) the myoelectric potential indicated by the acquired information on the myoelectric potential and (ii) the estimated myoelectric potential.

3. The information processing apparatus according to claim 1, wherein
the processor is configured to output a timing at which the difference satisfies a predetermined condition.

4. The information processing apparatus according to claim 2, wherein
the processor is configured to output a timing at which the difference satisfies a predetermined condition.

5. The information processing apparatus according to claim 1, further comprising:
an output device that is attachable to a part of a body of a user, the output device being configured to stimulate the part, wherein
the processor is configured to control the output device to stimulate a part corresponding to the muscle with a strength corresponding to a magnitude of the difference.

6. The information processing apparatus according to claim 2, further comprising:
an output device that is attachable to a part of a body of a user, the output device being configured to stimulate the part, wherein
the processor is configured to control the output device to stimulate a part corresponding to the muscle with a strength corresponding to a magnitude of the difference.

7. The information processing apparatus according to claim 3, further comprising:
an output device that is attachable to a part of a body of a user, the output device being configured to stimulate the part, wherein
the processor is configured to control the output device to stimulate a part corresponding to the muscle with a strength corresponding to a magnitude of the difference.

8. The information processing apparatus according to claim 4, further comprising:
an output device that is attachable to a part of a body of a user, the output device being configured to stimulate the part, wherein
the processor is configured to control the output device to stimulate a part corresponding to the muscle with a strength corresponding to a magnitude of the difference.

9. An information processing apparatus comprising:
a processor configured to
acquire information on a posture of a body,
acquires information on a myoelectric potential on a surface of the body,
specify a movement of the body based on the acquired information on the posture,
estimate a muscle activity state required for a muscle to implement the specified movement, and
output information indicating a difference between (i) a muscle activity state determined based on the myoelectric potential and (ii) the estimated muscle activity state.

10. A non-transitory computer readable medium storing a program that causes a computer comprising a processor to execute information processing, the information processing comprising:
acquiring the information on a posture of a body;
acquiring information on a myoelectric potential measured from a surface of the body;
specifying a movement of the body based on the acquired information on the posture;
estimating a muscle activity state required for a muscle to implement the specified movement; and
outputting information indicating a difference between (i) a muscle activity state determined based on the myoelectric potential and (ii) the estimated muscle activity state.
